(19) United States
(12) Patent Application Publication
(10) Pub. No.: US 2009/0239255 A1
(43) Pub. Date: Sep. 24, 2009

(54) Odontogenic ameloblast-associated protein as a tumor biomarker

(76) Inventors: Daniel P. Kestler, Maryville, TN (US); Alan Solomon, Knoxville, TN (US); James S. Foster, Knoxville, TN (US); Charles L. Murphy, Norris, TN (US)

Correspondence Address: HOWARD EISENBERG, ESQ., 1220 LIMBERLOST LANE, GLADWYNE, PA, 19035, US

(21) Appl. No.: 12/383,009

(22) Filed: Mar. 19, 2009

Related U.S. Application Data
(60) Provisional application No. 61/038,325, filed on Mar. 20, 2008.

(51) Int. Cl. C12Q 1/02

(52) U.S. Cl. 435/29

(57) ABSTRACT

Odontogenic ameloblast-associated (ODAM) protein may be used as an effective biomarker in humans for various cancers, especially those of epithelial origin, such as breast, lung, and stomach. The ODAM protein may be detected in cancerous cells. Additionally, the blood of patients with such cancers contains high titers of anti-ODAM antibodies.

This application claims the benefit of pending Provisional Patent Application Ser. No. 61/038,325, filed Mar. 20, 2008, which application is incorporated herein in its entirety by reference.

### FIELD OF THE INVENTION

The present invention pertains to the field of detection, diagnosis, prognosis, and management of cancer. Specifically, the present invention pertains to the field of using a substance produced by the body when cancer is present, referred to as a “tumor biomarker”, to aid in the diagnosis, prognosis, management, or treatment of individuals at risk of or afflicted with a cancer.

### BACKGROUND OF THE INVENTION

A tumor biomarker is a substance that can be detected in the body when cancer is present. The tumor biomarker may be produced by the cancer cells or may be produced by other cells of the body in response to the presence of cancer. The tumor biomarker may be found in a bodily fluid, such as blood or urine, or may be found in bodily tissues, such as in a tumor or in normal tissue.

Tumor biomarkers may be divided into two groups: cancer-associated and tissue-specific markers. The first is related to the presence of a certain specific cancerous tissue in the body. This type of marker generally is produced by the tumor itself. Examples of cancer-associated tumor biomarkers include the CEAs (carcinoembryonic antigens), certain of which are associated with gastric and colorectal cancer (CA 19-9), ovarian cancer (CA125), and breast cancer (CA 27-29).

Tissue-specific tumor biomarkers are not related specifically to the tumor itself but are generally present at elevated levels when a specific tissue is involved by the cancer. A well-known example of a tissue specific tumor biomarker is prostate specific antigen (PSA). Increases in PSA are associated with benign as well as malignant disorders of the prostate and, therefore, are not necessarily pathognomonic for cancer. Nonetheless, very high PSA levels are suggestive of prostate cancer and indicate the need to do further testing to determine whether the cause of the elevation in PSA levels is due to cancer or to another cause.

Tumor biomarkers are used in the detection, diagnosis, prognosis, and management of cancer. In general, an abnormal level of a tumor biomarker may suggest cancer. However, this finding alone is typically not sufficient to diagnose cancer, which requires a biopsy of the suspected lesion and its review by a pathologist.

Tumor biomarkers are often useful in early detection of or screening for cancer, that is, detecting the presence of a cancer before the onset of clinical signs or symptoms. An example of a tumor biomarker that is used in this way is PSA. Tumor biomarkers may also be useful in determining prognosis of a cancer. Very high levels of a marker may indicate that a cancer is more aggressive than would be the case if the marker was only slightly elevated. Tumor biomarkers are also useful in determining the effectiveness in treatment of a cancer. A decrease in level of the tumor biomarker or a return to a normal level following the initiation of treatment generally indicates that the tumor is responding to the treatment. Conversely, an increase in level of the tumor biomarker following treatment suggests that the treatment is ineffective. Tumor biomarkers are also useful in determining recurrence of a cancer following successful treatment of the cancer.

The term “diagnosis” when pertaining to the use of tumor biomarkers is used herein to refer to the use of a tumor biomarker, by itself or in conjunction with other medical tests, for early diagnosis or screening for cancer or as an aid to diagnosis of a cancer. The term “prognosis” when pertaining to the use of tumor biomarkers is used herein to refer to the use of a tumor biomarker, by itself or in conjunction with other medical tests, to determine how aggressive a cancer is likely to be in an individual. The term “monitor treatment” when pertaining to the use of tumor biomarkers is used herein to refer to the use of a tumor biomarker, by itself or in conjunction with other medical tests, to determine the effectiveness of treatment of a cancer. The term “recurrence” when pertaining to the use of tumor biomarkers is used herein to refer to the use of a tumor biomarker, by itself, or in conjunction with other medical tests, to determine if a cancer has recurred following treatment to which the cancer has previously favorably responded.

The term “management” when pertaining to the use of tumor biomarkers, is used herein to refer to the use of a tumor biomarker, by itself or in conjunction with other medical tests, following the diagnosis of a tumor in an individual. Thus, management includes prognosis, monitoring treatment, and assessing recurrence.

Odontogenic ameloblast-associated protein (ODAM) is a polypeptide that contains 279 amino acids and is designated by the NCBI (National Center for Biotechnology Information) as reference sequence: NP_{—}060325.3. Although transcripts from a gene encoding ODAM have been detected in human gastric cancer as well as in several normal mammalian tissues, the ODAM protein itself had never been found expressed until the publication by Solomon et al, Journal of Laboratory and Clinical Medicine, 142:348-355 (2003), which reported that amyloid associated with calcifying epithelial odontogenic tumors (CEOTs) contained a 46-residue amino acid polypeptide that was identified as being a portion of ODAM. This was the first evidence that the ODAM protein itself was actually expressed by human cells.

### DESCRIPTION OF THE INVENTION

The inventors have discovered that the ODAM protein can be detected in human epithelial cancers, including those of breast, lung, and gastric origin. This discovery was especially notable because, prior to the present invention, the ODAM protein had never been demonstrated to be expressed by normal or diseased human cells. The inventors also discovered that anti-ODAM antibodies are present in the serum of patients suffering from these same cancers.

In one embodiment, the invention is a method for aiding in the diagnosis of an individual at risk of or suspected of having a cancer or in the management of an individual known to have a cancer. According to this embodiment of the invention, the presence of the ODAM protein in a bodily tissue or fluid or the presence of anti-ODAM antibodies in a bodily fluid such as serum is determined. When the presence of ODAM or anti-ODAM antibodies are detected, this finding can be an aid in cancer diagnosis as well as the means to determine prognosis, efficacy of treatment, or recurrence.

The cancer for which the method of the invention is applicable is any cancer that is associated with increased expression of ODAM compared to the expression of ODAM in individuals not suffering from cancer. Typically, such cancers are epithelial in origin, such as adenocarcinomas. Examples of cancers for which the method of the invention is applicable include cancers of the breast, lung, and stomach.

In a particular embodiment, the invention is a method to aid in the diagnosis of a cancer. According to this embodiment of the invention, a sample of a bodily tissue or fluid, such as blood or urine, is obtained from an individual not known to have a cancer and the presence or absence of ODAM or anti-ODAM antibodies is determined in the sample. If either is present, then the level of ODAM or anti-ODAM antibodies may be determined. The presence of ODAM or anti-ODAM antibodies in the sample may be used to screen individuals to aid in early diagnosis of cancer. Following the determination that ODAM or anti-ODAM antibodies are present in the sample, additional tests may be employed to verify the presence of cancer and the site of origin.

In a particular embodiment, the invention is a method to aid in the management of an individual known to have a cancer. The presence and/or level of ODAM within a tumor or the presence and/or level of anti-ODAM antibodies in serum may be utilized to determine a prognosis, to monitor response to treatment, or to monitor the presence or recurrence of a cancer following treatment.

Tissue or fluid for analysis for the presence of ODAM or anti-ODAM antibodies may be obtained by any method by which a particular tissue or fluid may be obtained. For example, tissue for analysis may be obtained by biopsy, aspiration, or scraping, and blood may be obtained by venipuncture.

Determination of the presence of ODAM in a bodily tissue or fluid according to the method of the invention may be by any method by which it can be demonstrated that ODAM is present in the tissue or fluid. Its presence in cells may be determined immunohistochemically utilizing ODAM specific polyclonal or monoclonal antibodies. Its presence in body fluids such as blood may be determined, for example, by an ELISA based immunoassay using ODAM specific antibodies and recombinant or synthetic ODAM peptides.

Any method that is suitable for determining the presence of an antibody in a biologic sample is suitable for the method of the invention. Examples of methods for determining the presence of anti-ODAM antibodies in blood or other bodily fluids include Western blot and ELISA assay. Anti-ODAM antibodies that bind to a particular portion of the ODAM protein may be generated in a laboratory by known techniques, and such antibodies may be used for determining the presence of ODAM. The gene for human ODAM contains 10 exons that encode the ODAM protein as follows. Exon 1 encodes amino acids 1-17. Exon 2 encodes amino acids 18-31. Exon 3 encodes amino acids 32-48. Exon 4 encodes amino acids 49-125. Exon 5 encodes amino acids 126-141. Exon 6 encodes amino acids 142-176. Exon 7 encodes amino acids 177-192. Exon 8 encodes amino acids 193-216. Exon 9 encodes amino acids 217-270. Exon 10 encodes amino acids 271-279. Thus, anti-ODAM antibodies of the invention may bind to any one or more of these portions of the ODAM polypeptide.

The invention is further illustrated in the following non-limiting examples.

### EXAMPLE 1

### Generation of Anti-ODAM Antibodies

A polyclonal antiserum was obtained from a rabbit immunized with a 12-mer peptide containing ODAM amino acid residues 67-78. In addition, two IgG murine monoclonal antibodies (mAb) were generated. One of the mAbs, designated 5A-1, resulted from immunizing mice with a peptide encompassing residues 52 to 90 of ODAM, encoded by exon 4. The second mAb, designated 8B-4, was produced by immunizing mice with a 153-mer polypeptide (FLJ20513) containing the amino acids 126-279 encoded by exons 5 to 10.

The 8B-4 mAb immune-precipitated polypeptides containing amino acid sequences encoded by exons 2-10 and by exons 5-10 recombinant ODAM runoff translation products. As expected, the 5A-1 mAb did not react with the FLJ20513 polypeptide. In order to localize the region of ODAM recognized by 8B-4, the reactivity of this mAb was tested by Western blotting with proteolytically derived recombinant ODAM fragments spanning residues 178-219, 217-279, and 223-279. The 8B-4 antibody reacted only with the 178-279 component, indicating that the epitope was located between ODAM residues 178 and 216.

### EXAMPLE 2

### ODAM Expression by Normal and Malignant Dental Tissue

The anti-ODAM mab 5A-1 immunostained CEOT and ameloblastoma cells, as well as dental epithelial cells present in unerupted human tooth follicles and ameloblasts in 10-day-old mouse teeth. Similar results were obtained with the polyclonal anti-ODAM antiserum.

### EXAMPLE 3

### ODAM Expression by Non-Dental Normal Tissue

Hybridization of a 70-member human tissue array, using as a probe 32P-labeled cDNA that encodes the FLJ20513 polypeptide (ODAM amino acids 126-279), revealed a strong signal with tracheal and salivary gland-derived RNA, and a weak detection of stomach and fetal lung. ODAM expression in the two major sites also was evidenced by the demonstration that the epithelial cells were immunostained by mAb 8B-4, as well as by mAb 5A-1 and the polyclonal anti-ODAM antiserum. Immunohistochemical analysis of 35 other normal tissues revealed comparable immunostaining of the bronchus and, to a lesser extent, ovary, bladder, ureter, endometrium, and skin.

### EXAMPLE 4

### ODAM Expression by Non-Dental Malignant Tissue

Utilizing the probe of Example 3, human tumor tissue-specific arrays were analyzed and moderate to intense immunostaining was found in the nucleus and/or cytoplasm with the mAb 8B-4 in 46 of 60 breast, 48 of 60 gastric, and in 3 of 6 lung specimens contained in the arrays. The reactivity could be inhibited in the presence of the immunogen. Comparable results were obtained using mAb 5A-1.

The presence of ODAM within neoplastic breast cells was also shown by in situ hybridization using a biotin-labeled oligonucleotide probe specific for exons 2-10 encoded ODAM protein. This reactivity could be diminished by competition with the corresponding unlabeled specific ODAM oligonucleotide, but not with a non-corresponding ODAM antisense oligonucleotide. In contrast, the probe failed to hybridize with normal breast tissue. Further studies confirmed the presence of ODAM expression in gastric cancer as it was demonstrated immunohistochemically that anti-ODAM mAb 8B-4 immunostained, with varying degrees of intensity, all but 14 specimens in a 60-member human gastric tumor array. The detection of ODAM expression in other epithelial malignancies, e.g. breast and lung (as well as gastric cancers), indicates that ODAM is up-regulated in these cancers.

### EXAMPLE 5

### Serologic Evidence for ODAM

Antibodies to ODAM were found to be present in human sera, as evidenced by ELISA using recombinant ODAM protein as the solid phase antigen. Immunoassay of archival specimens from 72 females with breast cancer revealed that 25 (35%) had anti-ODAM IgG antibodies in titers higher than 2 standard deviations above the mean values of 45 out of 48 specimens obtained from normal women. The difference between the diseased and healthy groups was statistically significant, as determined by both standard and asymmetric t-test analyses (p<0.001). The present of anti-ODAM autoantibodies in patient sera also was demonstrated when these molecules immune-precipitated recombinant ODAM. Similarly, analyses of sera from 63 individuals with lung cancer and from 7 individuals with gastric cancer revealed that approximately 30% had anti-ODAM IgG antibody reactivity against recombinant ODAM>2 S.D. above the mean values of 27 normal subjects.

Autoantibodies are often found in sera of patients with malignancies and the titres of these antibodies have been correlated to survival and other clinicopathological parameters. Thus, the discovery as indicated herein of higher titer serum anti-ODAM antibodies in certain patients with epithelial cancers, such as breast, lung, and gastric, indicates that anti-ODAM antibodies may be used as a diagnostic and prognostic biomarker for patients with these and other epithelial types of malignancies.

Further modifications, uses, and applications of the invention described herein will be apparent to those skilled in the art. It is intended that such modifications be encompassed in the following claims.

1. A method for aiding in the diagnosis of an individual at risk of or suspected of having a cancer or in the management of an individual known to have a cancer comprising obtaining a bodily tissue or fluid sample of the individual and determining the presence or absence of odontogenic ameloblast-associated protein (ODAM) or of anti-ODAM antibodies in the bodily tissue or fluid sample, wherein the presence or absence and/or level of ODAM or anti-ODAM antibodies in the sample is correlated with presence, absence, or severity of the cancer in the individual.

2. The method of claim 1 wherein the cancer is an epithelial cancer.

3. The method of claim 2 wherein the epithelial cancer is a breast, lung, or gastric cancer.

4. The method of claim 1 wherein a fluid sample is obtained and the presence or absence of anti-ODAM antibodies is determined.

5. The method of claim 4 wherein the fluid sample is blood.

6. The method of claim 1 wherein a tissue sample is obtained and the presence or absence of the ODAM protein is determined.

7. The method of claim 6 wherein the tissue sample is of breast, lung, or stomach.

8. The method of claim 1 wherein the individual is not known to have a cancer.

9. The method of claim 1 wherein the individual is known to have a cancer.

10. The method of claim 9 wherein the cancer is an epithelial cancer.

11. The method of claim 10 wherein the cancer is breast, lung, or gastric.
